(19) United States
(12) Patent Application Publication
(10) Pub. No.: US 2020/0289090 A1
(43) Pub. Date: Sep. 17, 2020

(54) ULTRASONIC PROBE AND METHOD FOR MANUFACTURING SAME

(71) Applicant: Hitachi, Ltd., Tokyo (JP)

(72) Inventors: Takayuki Iwashita, Chiyoda-ku, Tokyo (JP); Takashi Fujii, Chiyoda-ku, Tokyo (JP); Kazuho Yoshimura, Chiyoda-ku, Tokyo (JP); Toru Watanabe, Chiyoda-ku, Tokyo (JP)

(73) Assignee: Hitachi, Ltd., Tokyo (JP)

(21) Appl. No.: 16/651,417

(22) PCT Filed: Dec. 26, 2018
(86) PCT No.: PCT/JP2018/047884

§ 371 Date: Mar. 27, 2020

(30) Foreign Application Priority Data
Mar. 15, 2018 (JP) 2018-048230

(51) Int. Cl. A61B 8/00; A61B 8/08; B29C 65/48

(52) U.S. Cl. CPC A61B 8/4455; A61B 8/0866; A61B 8/4494; B29K 2667/003; B29C 65/48; A61B 2562/12; A61B 8/483

(57) ABSTRACT

A second stacked product is configured with a flexible wiring sheet, and a stacked-element array supported by the flexible wiring sheet. To a biological-body side of the stacked-element array, a ground film is bonded, whereby a structurally reinforced third stacked product is produced. Then, the third stacked product is bent, whereby a curved stacked product is produced. In the bending procedure, in the ground film, a plurality of extension parts arranged along the θ direction are automatically formed.

### TECHNICAL FIELD

The present disclosure relates to an ultrasonic probe, and particularly, to an ultrasonic probe for three-dimensional diagnosis and to a method for manufacturing the ultrasonic probe.

### BACKGROUND

Ultrasonic diagnostic devices for performing three-dimensional diagnosis have been spreading. In such ultrasonic diagnostic devices, as ultrasonic probes, so-called 3D probes are used. For example, 3D probes which can be used in obstetrics departments have convex forms. They are called convex 3D probes (see Patent Document 1). Such a 3D probe has a two-dimensional transducer element array composed of a plurality of transducer elements arranged two-dimensionally along the convex surface. Ultrasonic beams are generated by the two-dimensional transducer element array, and the ultrasonic beams are used for two-dimensional scanning. As a result, volume data are acquired. The two-dimensional transducer element array may be composed of, for example, hundreds, thousands, tens of thousands, or more transducer elements.

### CITATION LIST

### Patent Literature

Patent Document 1: WO2005/053863

### SUMMARY

### Technical Problem

In a convex 3D probe, if necessary, a backing is provided, as a member for absorbing or attenuating ultrasonic waves radiated backward to the non-biological-body side of the two-dimensional transducer element array. Inside the backing, a plurality of signal lines (a plurality of leads) individually connected to the plurality of transducer elements are provided. Meanwhile, on the biological-body side of the two-dimensional transducer element array, a matching element array having conductivity is provided. From the plurality of transducer elements constituting the two-dimensional transducer element array and the plurality of matching elements constituting the two-dimensional matching element array, a plurality of stacked elements are configured. The plurality of stacked elements are covered with a ground electrode.

Using a ground film as the ground electrode can be considered. For example, a ground film is composed of a flexible resin sheet, and a conductive layer is provided on the non-biological-body side of the flexible resin sheet. When such a ground film is bonded to the plurality of stacked elements, it is feared that it will be easier for vibration to propagate between the stacked elements through the ground film. In the convex 3D probe, the two-dimensional transducer element array spreads out in the curvature direction, and in that direction, a number of transducer elements are arranged. It is desirable to reduce unnecessary vibration propagation between the stacked elements, at least in the curvature direction.

Meanwhile, it can be considered that in the procedure of producing a convex 3D probe, two-dimensional dicing is performed on a transducing layer and a matching layer stacked on a flexible wiring sheet such that a plurality of stacked elements are formed on the flexible wiring sheet, and the intermediate product obtained as a result is bent into a convex form. In this case, it is feared that if the plurality of stacked elements are bent in a state where they are supported by only the flexible wiring sheet, the directions of the plurality of stacked elements may become irregular. Also, it is feared that in the bending procedure, the adhesive may adhere to the ground surface exposed from each stacked element, or the exposed ground surface may be damaged.

An object of the present disclosure is to prevent occurrence of unnecessary vibration propagation between stacked elements in an ultrasonic probe, to the extent possible. Another object of the present disclosure is to prevent the directions of a plurality of stacked elements from becoming irregular, and to protect the ground surfaces of the individual stacked elements, in an ultrasonic probe manufacturing procedure.

### Solution to Problem

An ultrasonic probe according to the present disclosure is characterized by including a plurality of stacked elements arranged along a curved surface, and a ground film provided on the biological-body sides of the plurality of stacked elements, wherein the plurality of stacked elements are separated from one another by a plurality of groove parts extending along the curvature direction of the curved surface, and the ground film includes a plurality of adhesion parts bonded to the biological-body sides of the plurality of stacked elements, and a plurality of extension parts provided on the biological-body sides of the plurality of groove parts and extending along the curvature direction, and at least a part of each of the extension parts constitutes a thin part.

A method for manufacturing an ultrasonic probe according to the present disclosure is characterized by including a step of performing dicing on a first stacked product including a flexible wiring sheet, a transducing layer, and a matching layer, thereby producing a second stacked product including the flexible wiring sheet and a plurality of stacked elements supported by the flexible wiring sheet; a step of bonding a ground film on the biological-body sides of the plurality of stacked elements, thereby producing a third stacked product; a step of pressing the third stacked product against a convex curved surface of a backing member, thereby producing a curved stacked product; and a step of disposing a transducer assembly including the curved stacked product and the backing member inside a probe case.

### BRIEF DESCRIPTION OF DRAWINGS

FIG. 1 is a conceptual view illustrating an ultrasonic probe according to an embodiment.

FIG. 2 is a cross-sectional view illustrating a transducer assembly.

FIG. 3 is a first enlarged cross-sectional view illustrating a part of the transducer assembly.

FIG. 4 is a second enlarged cross-sectional view illustrating another part of the transducer assembly.

FIG. 5 is a flow chart illustrating a method for manufacturing the ultrasonic probe according to the embodiment.

FIG. 6 is a view illustrating a step of producing a first stacked product.

FIG. 7 is a view illustrating a step of producing a second stacked product.

FIG. 8 is a view illustrating a step of producing a third stacked product.

FIG. 9 is a view illustrating a flexible wiring sheet.

FIG. 10 is a view illustrating an example of a ground film positioning method.

FIG. 11 is a perspective view illustrating the transducer assembly assembled.

### DESCRIPTION OF EMBODIMENTS

Hereinafter, an embodiment will be described by reference to the drawings.

### (1) Outline of Embodiment

An ultrasonic probe according to the embodiment includes a plurality of stacked elements arranged two-dimensionally along a curved surface, and a ground film provided on the biological-body sides of the plurality of stacked elements. The plurality of stacked elements are separated from one another by a plurality of groove parts extending along the curvature direction of a curved surface. The ground film includes a plurality of adhesion parts bonded to the biological-body sides of the plurality of stacked elements, and a plurality of extension parts provided on the biological-body side of the plurality of groove parts and extending along the curvature direction. At least a part of each of the extension parts constitutes a thin part.

According to the above-described configuration, the ground film includes the plurality of extension parts extending in the curvature direction, and at least a part of each of the extension parts constitutes a thin part. In other words, each of the extension parts has a part weak in physical joining. Therefore, vibration propagation between the stacked elements through the ground film is reduced. The thin parts are parts thinner than the original thickness of the ground film or the thickness of the adhesion parts. For example, parts thinner than the original thickness by 10%, 20%, or more can be referred to as the thin parts. In the embodiment, the individual adhesion parts can be referred to as non-extension parts or predetermined-thickness parts. The concept of adhesion includes fixation. The adhesion parts can also be referred to as fixation parts. Each extension part is a part which is formed in the procedure of extending the ground film, or a part which is formed before extending the ground film.

In the embodiment, each adhesion part has a uniform thickness in the curvature direction, and the thickness of each thin part is smaller than the uniform thickness. Each adhesion part is a part through which an ultrasonic wave directed to a biological body or an ultrasonic wave from the biological body propagates. Therefore, when the adhesion parts have a uniform thickness, it is possible to suppress disturbance of ultrasonic waves during propagation. Practically, in the case where any change in the thickness is not visually recognized, or in the case where there is a change in thickness but the thickness change is practically negligible, it can be said that the thickness is uniform.

In the embodiment, a flexible wiring sheet supporting the plurality of stacked elements is included, and the two end parts of the ground film in the curvature direction are bonded to the respective end parts of the flexible wiring sheet in the curvature direction. According to this configuration, all of the plurality of stacked elements are interposed between the flexible wiring sheet and the ground film. Therefore, the plurality of stacked elements can be structurally reinforced. In the embodiment, on both end parts of the flexible wiring sheet, a plurality of ground terminals are provided, and the ground terminals are electrically connected to a conductive layer of the ground film.

A method for manufacturing an ultrasonic probe according to the embodiment includes a step of performing two-dimensional dicing on a first stacked product including a flexible wiring sheet, a transducing layer, and a matching layer, thereby producing a second stacked product including the flexible wiring sheet and a plurality of stacked elements supported by the flexible wiring sheet; a step of bonding a ground film to the biological-body sides of the plurality of stacked elements, thereby producing a third stacked product; a step of pressing the third stacked product against a convex curved surface of a backing member, thereby producing a curved stacked product; and a step of disposing a transducer assembly including the curved stacked product and the backing member inside a probe case.

According to this configuration, after the ground film is bonded to the plurality of stacked elements, the third stacked product is bent. Therefore, it is possible to prevent the directions of the plurality of stacked elements from becoming irregular. Further, since the ground surfaces of the individual stacked elements are not exposed in the bending procedure, it is possible to protect the individual ground surfaces.

In the embodiment, in the procedure of bending the third stacked product, in the ground film, a plurality of non-extension parts and a plurality of extension parts are alternately arranged along the curvature direction of the curved stacked product, and at least a part of each of the extension parts constitutes a thin part. After the ground film is completely bonded to the plurality of stacked elements, if the third stacked product is bent, the plurality of non-extension parts and extension parts alternately arranged along the curvature direction will be automatically formed.

The method according to the embodiment further includes a step of filling a filling material in the lattice-shaped grooves spatially separating the plurality of stacked elements from one another, after the ground film is bonded to the biological-body sides of the plurality of stacked elements.

In the case of filling the filling material in the lattice-shaped grooves before bonding the ground film, the filling material may adhere to the ground surfaces of the plurality of transducer elements. However, according to the above-described method, it is possible to prevent occurrence of the above-mentioned problem. Filling with the filling material may be performed before bending or after bending. In general, the filling material is composed of a rubber material or the like which can be easily deformed. Therefore, even if the filling material is filled before deformation, it does not cause any problem in the procedure of bending the third stacked product.

### (2) Details of Embodiment

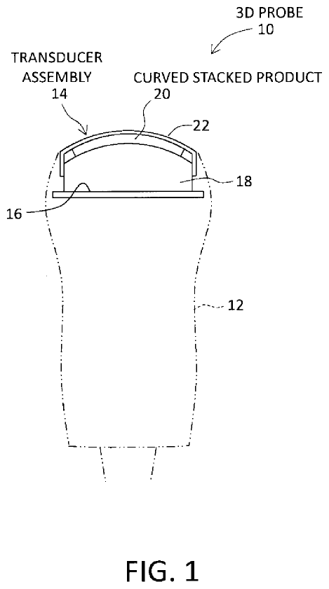

In FIG. 1, the ultrasonic probe according to the embodiment is schematically shown. The ultrasonic probe shown in the drawing is a 3D probe 10 for performing three-dimensional diagnosis. More specifically, the 3D probe 10 is, for example, a convex 3D probe for performing three-dimensional diagnosis on fetuses in obstetrics departments. The 3D probe 10 is a portable transceiver for transmitting and receiving ultrasonic waves, and can be connected to the main body of an ultrasonic diagnostic device (not shown in the drawing). The 3D probe 10 includes a two-dimensional transducer element array to be described below, the two-dimensional transducer element array generates ultrasonic beams, and the ultrasonic beams are used for two-dimensional scanning.

The 3D probe 10 includes a transducer assembly 14 disposed inside a probe case 12. The transducer assembly 14 includes a relay substrate 16, a backing 18 provided on the biological-body side of the relay substrate, a curved stacked product 20 provided on the biological-body side of the backing, and so on. The curved stacked product 20 is configured as a curved thin structure, and has a thickness, for example, in a range between 0.4 mm and 0.8 mm. On the biological-body side of the curved stacked product 20, a protective layer 22 is provided. The protective layer 22 may serve as an acoustic lens. The biological-body side surface of the protective layer 22 constitutes a wave transmission/reception surface, and the wave transmission/reception surface can be brought into contact with the surfaces of the abdominal areas of pregnant women.

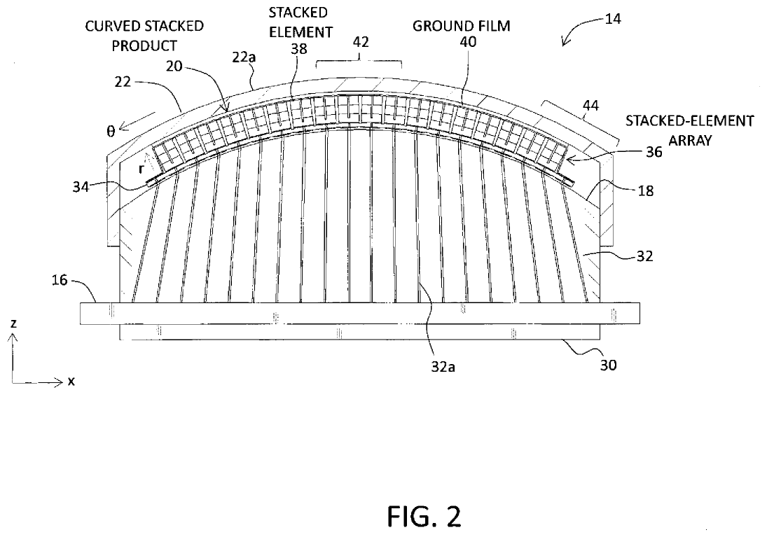

In FIG. 2, the transducer assembly 14 is shown. In FIG. 2, a z direction is a vertical direction. A first horizontal direction orthogonal to the z direction is an x direction, and a direction orthogonal to the z direction and the x direction is a y direction which is a second horizontal direction. A θ direction is the curvature direction of a curved surface. A direction extending from the center of curvature of the curved surface is an r direction. The z direction or the r direction is a direction to a biological body.

The relay substrate 16 is composed of, for example, a multilayer substrate for wiring. The relay substrate is also called an interposer. On the lower side of the relay substrate 16, an electronic circuit 30 is provided. The electronic circuit 30 is a circuit for channel reduction. The electronic circuit 30 includes a plurality of sub-beamformers, and is configured from, for example, six or eight ICs. On the biological-body side of the relay substrate 16, the backing 18 is provided. The backing 18 includes a backing member which is a base member, and a lead array 32 buried in the backing member. The lead array 32 is composed of a plurality of leads 32a arranged in the x direction and the y direction. Each lead 32a is a signal line for transmitting element transmission signals and element reception signals. The backing member is made of a material exhibiting an action of absorbing or scattering ultrasonic waves radiated backward.

The biological-body side surface of the backing 18 is a convex curved surface. The curved surface is a cylindrical surface, and has a constant curvature. To the curved surface, the curved stacked product 20 is bonded. The curved stacked product 20 includes a flexible wiring sheet 34, a stacked-element array 36 provided on the biological-body side of the flexible wiring sheet, and a ground film 40 provided on the biological-body side of the stacked-element array. The flexible wiring sheet 34 includes an insulating sheet, an upper surface electrode pad array formed on the upper surface (biological-body side surface) of the insulating sheet, and a lower surface electrode pad array formed on the lower surface (non-biological-body side surface) of the insulating sheet. The insulating sheet has an array of vias formed through the insulating sheet. The insulating sheet is made of, for example, a resin.

By the plurality of vias constituting the via array, the plurality of upper surface electrode pads constituting the upper surface electrode pad array are electrically connected to the plurality of lower surface electrode pads constituting the lower surface electrode pad array. Each via is filled with a conductive material. In the case where the individual vias are configured as through-holes, an adhesive may leak through them. However, the filling type vias do not cause the above-mentioned problem. Also, in the case where the individual vias have a property of becoming deformed in the z direction, even if the heights of the ends of the plurality of leads 32a are slightly uneven, when the plurality of upper surface electrode pads are connected to the individual leads, the plurality of vias can cope with the height unevenness.

The stacked-element array 36 is composed of, for example, tens of thousands of stacked elements 38 arranged in the θ direction and the y direction. The central axis of each stacked element 38 extends in the r direction. The stacked-element array 36 includes a hard backing element array, a transducer element array, and a matching element array, stacked up from the non-biological-body side toward the biological-body side. The matching element array is an array serving as a first matching layer.

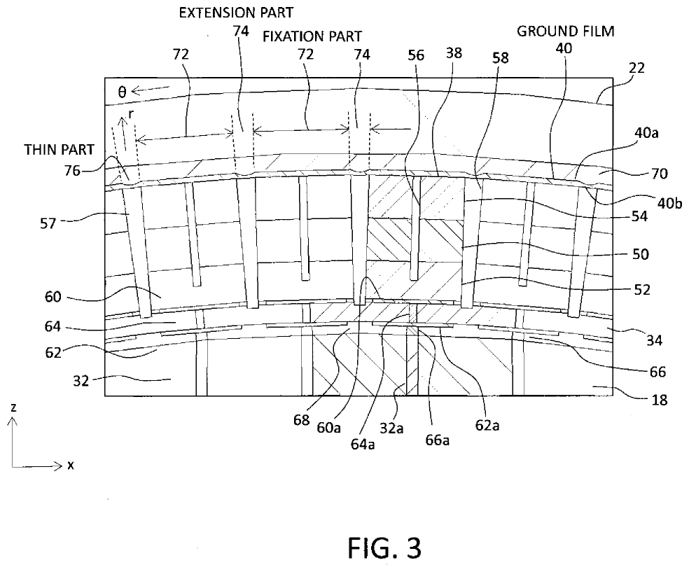

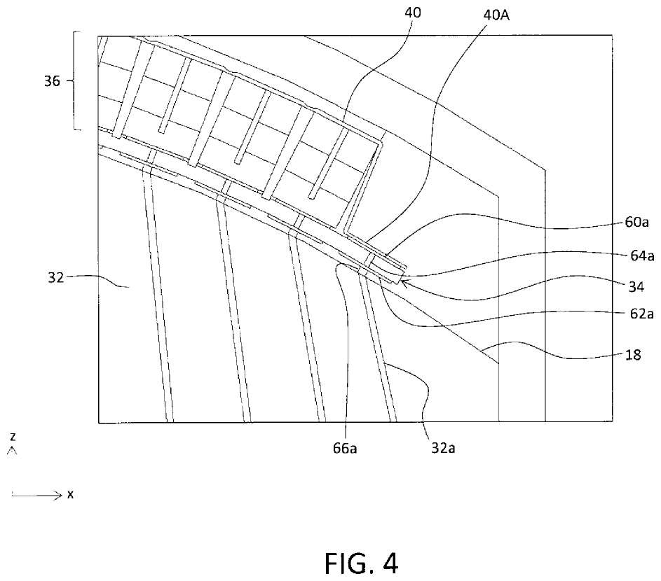

To the biological-body side of the stacked-element array 36, the ground film 40 is bonded. The ground film 40 is composed of a flexible film having an insulation property, and a thin conductive layer provided over the whole of the non-biological-body side of the flexible film. The film is made of a resin, and as an example of the resin, PET (polyethylene terephthalate) can be taken. The conductive layer is, for example, a deposited gold layer. The end parts of the ground film 40 are bonded to the respective end parts of the flexible wiring sheet 34. Incidentally, an enlarged cross-sectional view of a part denoted by reference symbol “42” in FIG. 2 is shown in FIG. 3. An enlarged cross-sectional view of a part denoted by reference symbol “44” in FIG. 2 is shown in FIG. 4.

In FIG. 3, the backing 18 includes the lead array 32, and the lead array is composed of the plurality of leads 32a arranged two-dimensionally. The biological-body side end part of the lead array 32 undergoes plating processing, whereby a contact array 66 is configured. The contact array 66 is composed of a plurality of contacts 66a arranged two-dimensionally. The flexible wiring sheet 34 includes an upper surface electrode pad array 60, a lower surface electrode pad array 62, and a via array 64. The upper surface electrode pad array 60 is composed of a plurality of upper surface electrode pads 60a arranged two-dimensionally. The lower surface electrode pad array 62 is composed of a plurality of lower surface electrode pads 62a arranged two-dimensionally. The via array 64 is composed of a plurality of vias 64a arranged two-dimensionally. In the transducer assembly, two members having a joining relation are bonded to each other by an adhesive. For example, the flexible wiring sheet 34 is bonded to the backing 18 by an adhesive 68. During bonding, if necessary, an insulating adhesive or a conductive adhesive may be used.

The flexible wiring sheet 34 is a member for supporting the plurality of stacked elements 38. In FIG. 3, the plurality of stacked elements 38 are arranged in the θ direction, such that they have a radial arrangement as seen from they direction. Each stacked element 38 is composed of a hard backing element 52, a transducer element 50, and a matching element 54. The hard backing element 52 has acoustic impedance greater than that of the transducer element 50, and serves as a resonance layer or a reflection layer. The hard backing element 52 has conductivity.

The transducer element 50 is made of PZT or the like which is a piezoelectric material. On the upper surface and lower surface of the transducer element 50, gold layers are deposited. The transducer element 50 exhibits an electromechanical transduction. The matching element 54 has acoustic impedance lower than that of the transducer element 50. The matching element 54 has conductivity. In each stacked element 38, a slit 56 for improving the electrical performance and acoustic characteristics of the corresponding stacked element is formed.

The plurality of stacked elements 38 are separated from one another by lattice-shaped grooves 57 as seen from the biological-body side. The lattice-shaped grooves 57 include a plurality of groove parts 58 arranged in the θ direction as shown in FIG. 3. In another aspect, the lattice-shaped grooves 57 include the plurality of groove parts 58 arranged in the y direction.

The ground film 40 has a plurality of adhesion parts 72 and a plurality of extension parts 74 alternately arranged along the θ direction. The individual adhesion parts 72 are parts fixed to the individual stacked elements 38 by bonding them to the biological-body side surfaces (ground surfaces) of the individual matching elements 54, respectively. In the embodiment, the adhesion parts 72 are parts that are firmly bonded. The individual extension parts 74 are parts which are automatically formed in a procedure of bending a stacked product to produce a curved stacked product as will be described below. In other words, in a bending procedure, between the inner side and outer side of the product which is bent, a difference in path length occurs, and in order to cope with the path length difference, the plurality of extension parts 74 are formed between the plurality of stacked elements 38. When attention is paid to the θ direction, the groove parts 58 exist between neighboring stacked elements 38, and the extension parts 74 are formed on the biological-body sides of the groove parts 58. Each groove part 58 has a form slightly spreading out toward the biological-body side. At least a part of each extension part 74 constitutes a thin part 76. In other words, each extension part has a part having a smaller thickness in the r direction. Most of each extension part 74 may be formed as a thin part 76.

Each adhesion part 72 is a part having a width in the r direction and having a uniform thickness, and is a non-extension part which basically is not extended in a bending procedure. The thin part 76 of each extension part has a thickness smaller than the thickness of each adhesion part 72. In the embodiment, the biological-body side surface of each thin part 76 is recessed toward the non-biological-body side, and the recessed part extends in the y direction. The non-biological-body side surface of each thin part 76 is flat.

The effect of reducing vibration propagation though the individual extension parts 74 by the individual thin parts 76 can be expected. In the θ direction, the plurality of thin parts 76 are configured with a stacked element pitch. Therefore, over the whole area in the θ direction, the above-mentioned effect can be expected. In other words, in the θ direction, an improvement in image quality can be expected. In the embodiment, in the bending procedure, the plurality of extension parts 74 are automatically formed. Therefore, it is unnecessary to provide a special step only for providing the plurality of extension parts 74. Since each adhesion part 72 is a predetermined-thickness part and has a uniform thickness equal to a design value, it is possible to prevent occurrence of disturbance in ultrasonic wave propagation in each adhesion part 72. Incidentally, in the embodiment, in the y direction, between the stacked elements 38, no thin part is formed. However, not only in the θ direction but also in the y direction, thin parts may be formed between the stacked elements 38.

As will be described below, after bonding of the ground film 40, the lattice-shaped grooves 57 are filled with the filling material before bending. The filling material is composed of a rubber-based material, such that the filling material does not hinder bending. On the biological-body side of the ground film 40, a single second matching layer 70 is provided. The second matching layer 70 also is composed of the rubber-based material, such that it does not hinder bending.

The rubber-based material excellently transmits ultrasonic waves in the ultrasonic wave traveling direction, but rarely transmits ultrasonic waves in the direction orthogonal to the ultrasonic wave traveling direction. Therefore, in the filling material and the second matching layer 70, ultrasonic wave propagation between the stacked elements is negligible. Incidentally, between the second matching layer 70 and the protective layer 22, a thin barrier film may be provided if necessary.

In FIG. 4, an enlarged view of an end part of the transducer assembly is shown. The stacked-element array 36 is provided on the flexible wiring sheet 34. In the θ direction, both end parts of the flexible wiring sheet 34 protrude from the stacked-element array 36. The stacked-element array 36 is covered with the ground film 40. In the θ direction, the end parts 40A of the ground film 40 are bonded to the respective end parts of the flexible wiring sheet 34. In FIG. 4, the conductive layer of the ground film 40 is connected to the ground leads 32a of the lead array 32 provided in the backing 18, through the upper surface electrode pads 60a, the vias 64a, the lower surface electrode pads 62a, and the contacts 66a. Actually, the plurality of ground leads are electrically connected to the conductive layer of the ground film 40. As a result, the electric resistance decreases.

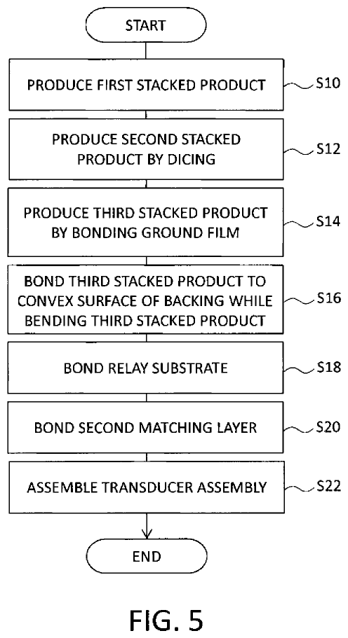

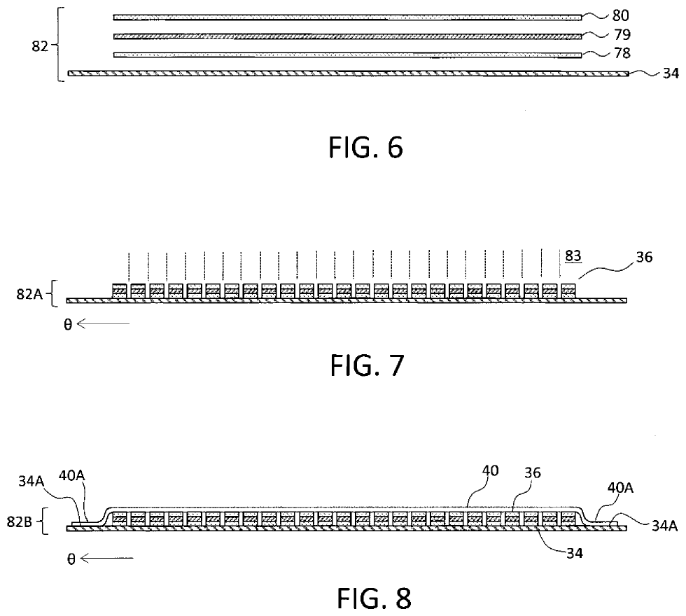

Now, the method for manufacturing an ultrasonic probe according to the embodiment will be described with reference to FIG. 6 and the subsequent drawings, mainly with reference to the flow chart shown in FIG. 5.

In STEP S10 shown in FIG. 5, a first stacked product is produced. Specifically, as shown in FIG. 6, on the flexible wiring sheet 34, a hard backing layer 78, a transducing layer 79, and a matching layer 80 are stacked and bonded to one another. As a result, a plate-like first stacked product 82 is produced. In STEP S12 shown in FIG. 5, a second stacked product is produced. Specifically, as shown in FIG. 7, the stacked-element array 36 is formed by performing two-dimensional dicing 83 on the first stacked product. During the two-dimensional dicing 83, the hard backing layer, the transducing layer, and the matching layer are cut, but the flexible wiring sheet 34 is not cut. As the result of the two-dimensional dicing, a second stacked product 82A is produced.

In STEP S14 shown in FIG. 5, the third stacked product is produced. Specifically, as shown in FIG. 8, the ground film 40 is bonded to the biological-body side of the stacked-element array 36. At this time, the end parts 40A of the ground film 40 in the θ direction are bonded to the respective end parts 34A of the flexible wiring sheet 34 in the θ direction. As a result, a third stacked product 82B is produced as an intermediate product before bending. In this stage, the gaps between the plurality of stacked elements; i.e., the lattice-shaped grooves are filled with the filling material. At this time, the third stacked product 82B is placed in a vacuum chamber. The filling material may be filled after bending.

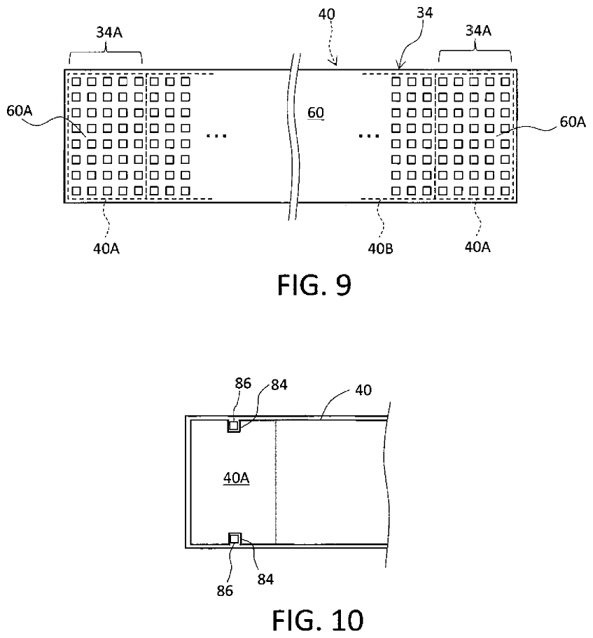

In FIG. 9, the upper surface (biological-body side surface) of the flexible wiring sheet 34 is shown. On the upper surface, the upper surface electrode pad array is formed. As described above, the end parts 40A of the ground film 40 are bonded to the respective end parts 34A of the flexible wiring sheet 34. Therefore, a plurality of upper surface electrode pads 60A of the upper surface electrode pad array 60 provided on both end parts 34A are connected to the conductive layer of the ground film 40. Reference symbol 40B indicates a part of the ground film 40 which is bonded to the stacked-element array.

As shown in FIG. 10, a plurality of notches 84 may be formed in both end parts 40A of the ground film 40, and the ground film 40 may be positioned such that the center of a plurality of specific electrode pads 86 coincides with the center of the plurality of notches 84.

In STEP S16 shown in FIG. 5, the third stacked product is bonded to the convex curved surface of the backing. Specifically, the third stacked product is pressed against the curved surface, thereby being bent. As a result, a curved stacked product is produced. Before bending of the third stacked product, in the ground film, a plurality of adhesion parts are formed along the θ direction. Each adhesion part is a part completely fixed to a corresponding stacked element, and is a part having a uniform thickness. Each adhesion part basically is not deformed during bending, and the thickness thereof is maintained. In the bending procedure, in the ground film, a plurality of extension parts are formed along the θ direction. Each extension part is a part extended in the θ direction by bending. At least a part of each extension part constitutes a thin part.

In STEP S18 shown in FIG. 5, a relay substrate is bonded to the backing. The relay substrate has an electronic circuit provided in advance. The electronic circuit may be provided on the relay substrate after the relay substrate is bonded to the backing. In STEP S20 shown in FIG. 5, a second matching layer is bonded to the biological-body side of the curved stacked product. Further, a protective layer is bonded to the biological-body side of the second matching layer. The order of STEP S18 and STEP S20 may be reversed, or these steps may be performed in parallel. In STEP S22 shown in FIG. 5, a transducer assembly is disposed inside a probe case.

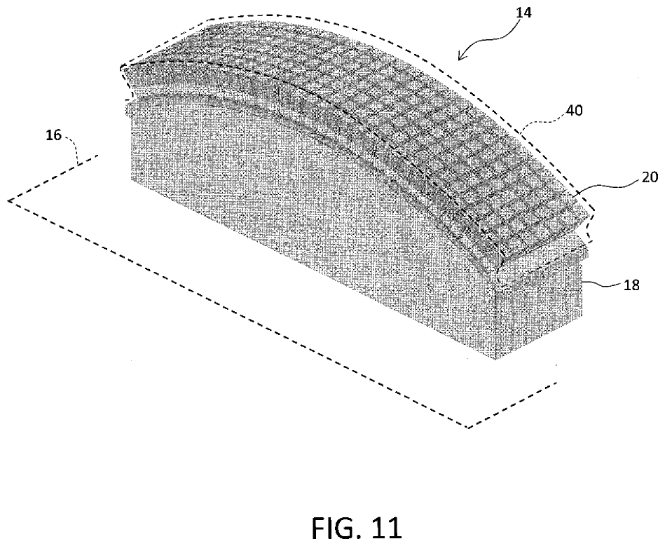

In FIG. 11, the assembled transducer assembly 14 is shown. On the biological-body side of the backing 18, there is the curved stacked product 20. The ground film 40 is shown by a broken line. Further, the relay substrate 16 is shown by another broken line.

According to the manufacturing method of the embodiment, the ground film is provided on the second stacked product before bending, such that the stacked-element array is interposed between the flexible wiring sheet and the ground film. Therefore, it is possible to structurally reinforce the stacked-element array, and particularly, in the bending procedure, the directions of the plurality of stacked elements are prevented from becoming irregular. Also, it becomes possible to protect the ground surfaces of the individual stacked elements in the bending procedure. Further, in the bending procedure, the plurality of extension parts can be automatically formed in the θ direction; i.e., the plurality of thin parts can be automatically formed. Therefore, an advantage that it is unnecessary to provide a special step for forming the plurality of extension part is obtained.

1. An ultrasonic probe comprising:
a plurality of stacked elements arranged two-dimensionally along a curved surface; and
a ground film provided on the biological-body sides of the plurality of stacked elements,
wherein the plurality of stacked elements are separated from one another by a plurality of groove parts arranged along the curvature direction of the curved surface, and
the ground film includes:
a plurality of adhesion parts bonded to the biological-body sides of the plurality of stacked elements; and
a plurality of extension parts provided on the biological-body sides of the plurality of groove parts and arranged along the curvature direction, and
at least a part of each of the extension parts constitutes a thin part.

2. The ultrasonic probe according to claim 1, wherein
each of the adhesion parts has a uniform thickness in the curvature direction, and
the thickness of the thin part is smaller than the uniform thickness.

3. The ultrasonic probe according to claim 1, further comprising:
a flexible wiring sheet supporting the plurality of stacked elements,
wherein the end parts of the ground film in the curvature direction are bonded to the respective end parts of the flexible wiring sheet in the curvature direction.

4. A method for manufacturing an ultrasonic probe, comprising:
a step of performing two-dimensional dicing on a first stacked product including a flexible wiring sheet, a transducing layer, and a matching layer, thereby producing a second stacked product including the flexible wiring sheet and a plurality of stacked elements supported by the flexible wiring sheet;
a step of bonding a ground film on the biological-body sides of the plurality of stacked elements, thereby producing a third stacked product;
a step of pressing the third stacked product against a convex curved surface of a backing member, thereby producing a curved stacked product; and
a step of disposing a transducer assembly including the curved stacked product and the backing member inside a probe case.

5. The manufacturing method according to claim 4, wherein
in the procedure of bending the third stacked product, in the ground film, a plurality of non-extension parts and a plurality of extension parts are formed alternately along the curvature direction of the curved stacked product, and at least a part of each of the extension parts constitutes a thin part.

6. The manufacturing method according to claim 4, further comprising:
a step of filling a filling material in lattice-shaped grooves spatially separating the plurality of stacked elements, after bonding the ground film to the biological-body sides of the plurality of stacked elements.
